(19) United States
(12) Patent Application Publication
(10) Pub. No.: US 2022/0111090 A1
(43) Pub. Date: Apr. 14, 2022

(54) DEVICE FOR USE WITH MEDICAL PRODUCTS WHICH CAN BE DISINFECTED BY MEANS OF A UV-C LIGHT TREATMENT

(71) Applicant: US SMART B.V., Delft (NL)

(72) Inventors: Thijs Joop KEA, Amsterdam (NL); Daan Jelmer HOEK, Amsterdam (NL); Cornelis Wilhelmus Johannes Maria LAUWERIJSSEN, SINT-MICHIELSGESTEL (NL)

(21) Appl. No.: 17/421,937

(22) PCT Filed: Jan. 6, 2020
(86) PCT No.: PCT/NL2020/050004

§ 371 Date: Jul. 9, 2021

(30) Foreign Application Priority Data
Jan. 10, 2019 (NL) 2022365

(51) Int. Cl. A61L 2/10

(52) U.S. Cl. CPC A61L 2/10; A61L 2202/14; A61L 2202/11

(57) ABSTRACT

The invention relates to a device for use with medical products which can be disinfected by means of a UV-C light treatment, which device is provided with:
at least one sensor for registering a UV-C light dose of the UV-C light treatment;
at least one activable reset unit which can perform a memory reset of a memory of the device;
at least one indicator;
at least one logic module which is operatively connected to the memory, the sensor, the reset unit and the indicator, wherein the logic module is configured to determine whether the product has been disinfected in a prescribed way on the basis of the UV-C dose registered by the sensor and to determine whether the reset unit has been activated, wherein, by means of the logic module, the memory can be placed in a first state, corresponding to a product disinfected in the prescribed way, and can be placed in a second state, corresponding to the memory reset by means of the activated reset unit, wherein the state of the memory is externally communicable by means of the indicator.

The invention relates to a device for use with medical products which can be disinfected by means of a UV-C light treatment.

One of the problems of medical products which can be disinfected by means of a UV-C light treatment, is the fact that it is not visible to a user whether the product has been disinfected.

The object of the invention is therefore to provide a device which can make it clear whether a medical product has been disinfected or has to be disinfected before it can be used with a patient or in a medical treatment.

This object is achieved by means of the device defined in claim 1.

The device is provided with:
at least one sensor for registering a UV-C light dose of a disinfecting UV-C light treatment of a medical product;
at least one activable reset unit which can perform a memory reset of a memory of the device;
at least one indicator;
at least one logic module which is operatively connected to the memory, the sensor, the reset unit and the indicator, wherein the logic module is configured to determine whether the product has been disinfected in the prescribed way on the basis of the UV-C dose registered by the sensor and to determine whether the reset unit has been activated, wherein, by means of the logic module, the memory can be placed in a first state, corresponding to a product disinfected in the prescribed way, and can be placed in a second state, corresponding to the memory reset by means of the activated reset unit, wherein the state of the memory is externally communicable by means of the indicator.

By means of the above-described device, and the state indicated by the indicator, it becomes clear to a user whether the medical product has been disinfected in the prescribed way in a disinfecting device by means of a UV-C treatment or has to be disinfected by means of a UV-C treatment. Specifically, a user is not able to determine whether a product has been disinfected by means of a UV-C treatment on the product per se, which insecurity is particularly undesirable in the case of medical products.

If a UV-C light dose of the disinfecting UV-C light treatment of the medical product is registered by means of the sensor and the logic module determines that the prescribed UV-C light dose has been received, the first state is input into the memory and this state can be communicated to a user by means of the indicator. The product is now ready for use in the medical operation to be performed. After use/application of the medical product, the activable reset unit will be activated, wherein the logic module will put the memory in the second state after the logic module has determined the memory reset by the activated reset unit, which second state corresponds to a reset of the memory. By means of the indicator of the device, this second state is communicated to the user.

The reset unit can be activated manually by a user after a medical operation has been performed with the product, for example by means of a manually activable switch on the reset unit or the like.

The activable reset unit may also comprise a receiver for (contactlessly/wirelessly) receiving a reset signal by means of which the memory can be placed in the second state, so that this status can be communicated via the indicator. The person noticing this status then becomes aware that the product has to be cleaned again by means of a disinfecting apparatus before the product can be used in a subsequent medical operation. The reset signal may be transmitted, for example, in an area of a tray or the like in which products have to be placed after a medical operation has been performed. This has the advantage that the user is not responsible for activating the second state himself, so that it is not possible for the user to forget to perform this operation. However, it is also possible for the receiver to receive the reset signal via a scan-type operation by the user after the product has been used.

Disinfecting by means of UV-C light offers a user-friendly method for disinfecting medical objects. If this light is dosed accurately, it is possible to disinfect up to 5-log (99.999% extermination) in a time period of 20 to 60 seconds. Disinfecting a medical object by means of UV-C light has minimal, if any, negative effects on the object to be disinfected. In addition, it is possible to perform disinfection at relatively low temperatures (15-40 degrees Celsius) and, due to the fact that no liquids or chemicals are used, the UV-C light disinfecting process is quick, relatively safe and reliable. As a result thereof, electronic medical apparatuses may be disinfected by means of the UV-C light disinfecting process.

In addition, the invention relates to a system which is provided with the above-described device and with an activator for activating the activable reset unit. In one aspect, the activator is a transmitter for transmitting the reset signal to a receiver of the device. The device is attached to or incorporated in a medical product.

The system may in addition be provided with a disinfecting device with which the device is able to communicate, for example during a disinfecting process to be performed using the disinfecting device. As a result thereof, it is possible to transmit a signal to the disinfecting device via the transceiver of the device, after the desired UV-C light dose registered by the device has been received, so that a control unit in the disinfecting device can decide to switch off the UV-C light-emitting light sources. In this way, the service life of the UV-C light-emitting light sources can be extended.

The above-described aspects will be explained below by means of exemplary embodiments in combination with the figures. However, the invention is not limited to the exemplary embodiment described below. Rather, a number of variants and modifications are possible, which also use the inventive idea and are thus covered by the scope of protection. In particular, the possibility is mentioned to combine the properties/aspects which have only been mentioned in the description and/or have been shown in the figures with the features of the claims, if compatible.

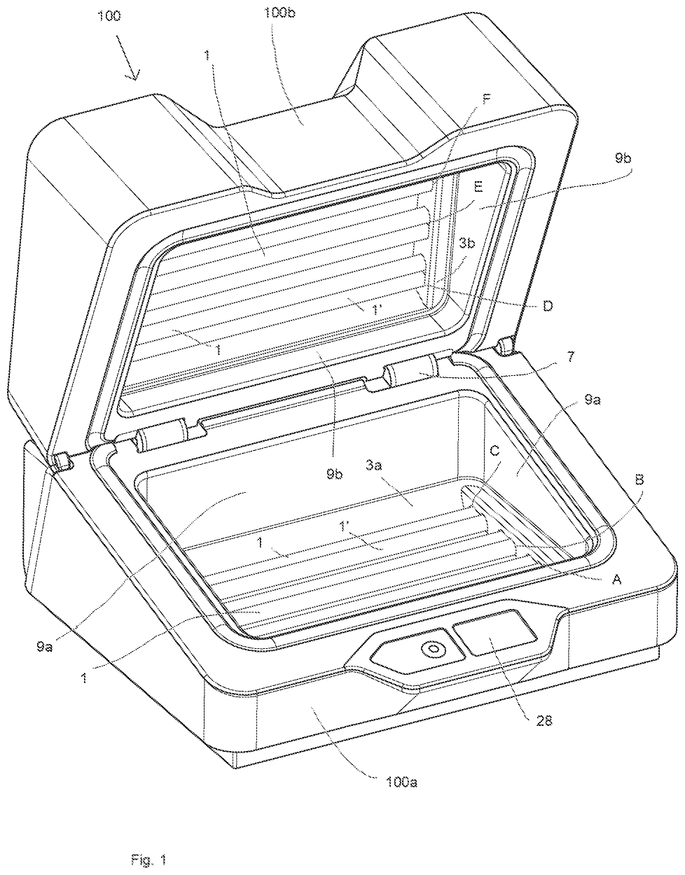

FIG. 1 shows a perspective view of a disinfecting device for disinfecting a product;

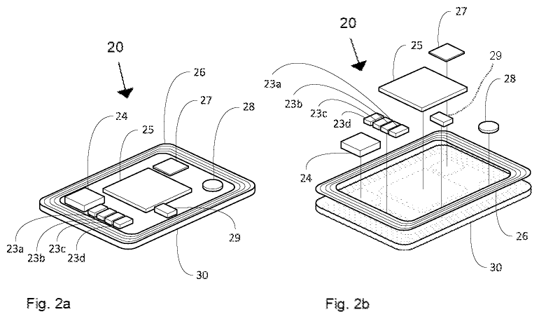

FIGS. 2a, b show a device according to the present invention;

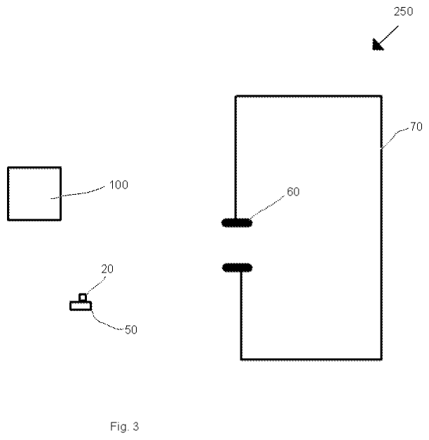

FIG. 3 shows a diagrammatic view of the disinfecting device illustrated in FIG. 1, and a system comprising a medical product provided with the device illustrated in FIG. 2 and an operating theatre door which is provided with an activator in the form of an activable transmitter.

In the figures, similar components are provided with identical reference numerals.

FIG. 1 shows a disinfecting device 100 for disinfecting a medical product, wherein the product or the products can be positioned, for example, on the flat glass panel 3a by a user for disinfecting purposes.

The disinfecting device 100 is provided with light sources by means of which UV-C light can be provided in the chamber. The light sources are arranged in pairs and every pair A-F in the illustrated example comprises a first lamp 1 and a second lamp 1′.

By means of the lamps 1, 1′, UV-C light can be emitted in a closable chamber of the disinfecting device 100. In operation, the maximum operating temperature in the disinfecting device 100 will at most be 50 degrees Celsius. In addition, the disinfecting device 100 does not use any liquids to disinfect. The disinfecting device 100 is therefore particularly suitable for disinfecting electronic medical apparatuses.

The disinfecting device is provided with a stationary part 100a and with a part 100b which is hingeable about a hinge pin via hinges 7 and which is movable between an open position in which the chamber is accessible and a closed position in which the chamber is closed. Both parts 100a, 100b form the housing of the disinfecting device and are provided with their own light sources 1, 1′. Each part 100a, 100b comprises light source pairs A, B, C; D, E, F which run parallel to each other. In the closed position, the object to be disinfected is placed between the light source pairs A, B, C; D, E, F, so that the top side and the bottom side of the object can be exposed to UV-C light during operation.

For health and/or safety reasons, disinfecting should only be performed in the closed position, that is to say in a closed chamber. The chamber is defined by walls, that is to say the glass panels 3a, 3b, and reflecting side walls 9a, 9b of the disinfecting device 100.

The disinfecting device 100 is furthermore provided with a display 28 by means of which, for example, inter alia the desired disinfecting programme can be chosen and the course of the disinfecting process can be followed.

FIGS. 2a, b show the device 20 as claimed. This device 20 may be incorporated in an outer surface of a medical product or it may be attached to an outer surface of a medical product. Although the device 20 is particularly suitable for medical products, the device 20 may also be used in combination with other products. The device 20 may, for example, be configured as a sticker, so that the bottom layer 30 is at least partly provided with an adhesive layer by means of which the device can be stuck onto a medical product 50, as is shown, for example, in FIG. 3.

By means of the device 20, a user is able to determine if a product 50 requires disinfection by means of a UV-C light treatment or has already undergone the prescribed disinfecting process. This information is used to determine if the medical object is safe for use on or with a patient.

The device 20 is provided with:
sensors 23a-d, wherein at least one sensor 23a is provided for registering a UV-C light dose of the UV-C light treatment;
a receiver 27 for (wirelessly/contactlessly) receiving a reset signal;
an indicator in the form of a display 25;
at least one logic module 24.

The logic module 24 is operatively connected to a memory 29, the sensors 23a-d, the receiver 27 and the indicator 25. The logic module 24 is configured to determine if the product 50 has been disinfected in a prescribed way on the basis of the UV-C dose registered by the sensor 23a, and to reset the memory 29 on the basis of the reset signal received by the receiver 27, wherein, by means of the logic module 24, the memory 29 can be placed in a first state, corresponding to a product disinfected in the prescribed way, and a second state, corresponding to a received reset signal, wherein the state of the memory 29 is externally communicable by means of the indicator 25. By means of the device 20, a user can quickly and reliably determine whether the medical product is suitable for use. The indicator 25 may present the first or the second state via the display so as to be immediately noticeable for a user. Instead thereof or in addition thereto, the first or the second state may in addition be readable, for example by means of a scanner by means of the indicator.

In addition, the receiver 27 may be a transceiver, so that it is also possible to transmit a signal by means of the device 20. The signal to be transmitted may, for example, be a product-identification item for, for example, the disinfecting device 100 illustrated in FIG. 1, so that this device 100 can be automatically set to the specifically prescribed UV-C light treatment for this product on the basis of the signal. Furthermore, the status and the progress of the UV-C light treatment process may be passed on to the disinfecting device via the transceiver. In this way, direct validation of the disinfecting of the product can immediately be achieved. In addition, the transmitting part of the transceiver may form the indicator for transmitting the first or the second state to an external apparatus which indicates to the user whether the product has been disinfected or not.

The receiver 27 may, in addition, be a manually activable reset unit (not shown).

The memory 29 is a non-volatile memory, so that the state of the memory cannot be lost.

The device 20 is provided with a power supply, for example a battery 28. The power supply may, in addition, be formed by a capacitor. In an alternative variant, the device 20 is provided with energy by means of an inductive coupling (not shown), so that the device 20 does not comprise its own power source or energy storage.

The logic module is configured to receive data, such as product data, via the receiver. In this way, a universal device can be used for many types of medical products, in which case specific information on the product can be supplied to the device after the device has been coupled to a medical product.

The device 20 may optionally be subjected to various other disinfecting or sterilisation techniques and determine, optionally by means of other sensors 23b-d, if the disinfecting and/or sterilising procedure performed using these techniques has taken place in the prescribed way. Consideration may be given to, for example, autoclaving and a UV-C disinfection, or a UV-C disinfection in combination with a thermal disinfection, plasma sterilisation, disinfecting wipes, hydrogen peroxide disinfection, a disinfection by means of formaldehyde and/or peracetic acid, or ultrasonic cleaning, etc.

The logic module 24 is configured to execute programmable logic in order to determine, by means of the logic module, whether a UV-C light disinfecting procedure has already been performed on a product or is required. By means of the programmable logic, the logic module 24 can also be used to determine, on the basis of the other sensors, if another disinfecting process or sterilisation has been performed or has to be performed before the indicator may indicate the first state. Various of the following parameters and sensor input values may be considered for this purpose, such as for example time, temperature, moisture, humidity, movement, for example a vibration, noise, pressure, pH value, location, glucose, oxygen and/or carbon dioxide.

In any case, in a minimal embodiment of the device, the UV-C dose is determined using the device as described above.

During the disinfecting or sterilising process for a medical instrument/apparatus, there are often various checkpoints at which information can be be registered and linked to the product by means of the device 20. An example of a number of steps where information can be exchanged with the device 20:

1. A safety inspection (leakage test) of the medical product
2. Confirmation whether disinfecting process has been performed on the medical product
3. Registration of use of the medical product
4. Registration of inclusion in the cleaning process of the medical product
5. Repetition from step 1

These steps are based on an optimum case, in which the product is stored only for a short length of time, if at all. Often, the product is packaged after step 2 disinfection and only used when it is needed for a patient.

In this exemplary process, the device 20 also offers the option to register all process steps, for example by means of a scanner and a barcode. During scanning, more information may be transferred than just the process step. There are very many situations and instances in a (similar) process where a medical apparatus or instrument is being used, is stored or does not have any registration instances for a prolonged period of time. In these kinds of instances, it is desirable for the device to actively communicate to users and supervising staff whether or not the apparatus is still sufficiently hygienic to be used responsibly on patients. As described above, the device 20 is able to register various activities around a medical product by means of the sensors 23a-d and determine on the basis thereof whether a certain type of disinfection is required because the apparatus has been contaminated/become less safe. The various activities may be stored in the device 20 or in an external system (not shown), which makes it possible to communicate, upon scanning, via the indicator whether the product can still be used or whether an operation has to be performed before use. This may also be communicated to a user visually, for example, via the display, e.g. by a cross or tick, so that the user knows whether the product may or may not be used with a patient. The external transmitter and the receiver 27 of the device 20 may operate using identification via radio waves, RFID.

FIG. 3 shows an exemplary configuration of the claimed system 250. The system comprises a device 20 attached to a product 50 and a transmitter 60 for transmitting a reset signal. In this example, the transmitter 60 is incorporated in a doorway of an operating theatre 70. However, it is also possible to configure the transmitter in such a way that the signals therefrom are detectable throughout the entire operating theatre 70 or that the transmitter is a portable scanner (not shown) by means of which a reset signal can be transmitted to the device manually. The transmitter 60 can be activated, for example, for transmitting the reset signal after an operation. The medical products which are provided with the device 20 are earmarked as no longer being suitable for use when the reset signal is received. In the example illustrated in FIG. 3, the transmitter 60 will be activated following an operation performed in the operating theatre, so that the receiver of the device 20 receives the reset signal when the product 50 is moved out of operating theatre, so that the second state is indicated by means of the indicator, which makes it clear to a user that the product has to be disinfected.

In addition, by providing transmitters in the form of so-called “gateways” throughout, for example, a hospital, it is possible to record where products are located. The unique codes of every device 20 are product-specific and can be read by the gateways. This provides logistical overview. In addition, a gateway at the exit of the hospital can prevent the undesired removal or theft of products or at least reduce it.

The battery 28 can provide the device 20 with power for several months. In addition, it is possible to opt for charging solutions. By means of wireless charging, the rechargeable battery 28 is for example charged by the disinfecting device during the moments of contact. In addition, the device 20 may comprise a solar cell (not shown), by means of which the device can be charged, inter alia, during the disinfecting cycle.

The device 20 may in addition be sealed, for example by means of a waterproof, UV(-C) permeable layer which ensures that the charging and information-communicating properties can pass through.

1-15. (canceled)

16. A device for use with medical products adapted to be disinfected by an ultraviolet C (UV-C) light treatment, the device comprising:
at least one sensor configured to register a UV-C light dose of a UV-C light treatment;
at least one activatable reset unit configured to perform a memory reset of a memory of the device;
at least one indicator; and
at least one logic module operatively coupled to each of the at least one sensor, the at least one activatable reset unit, the memory of the device, and the at least one indicator, wherein the at least one logic module is operable to:
determine whether medical products have been disinfected in a prescribed way on the basis of a UV-C dose registered by the at least one sensor and to determine whether the at least one activatable reset unit has been activated, wherein, by the at least one logic module, the memory is configured to be placed in a first state corresponding to a medical product disinfected in the prescribed way, and in a second state corresponding to the memory reset via the activated at least one activatable reset unit, wherein the state of the memory is externally communicated by the at least one indicator.

17. The device according to claim 16, wherein the at least one activatable reset unit comprises a receiver operable for receiving a reset signal to place the memory in the second state.

18. The device according to claim 16, further comprising a receiver.

19. The device according to claim 18, wherein the at least one logic module is operable for receiving data via the receiver.

20. The device according to claim 16, further comprising a power supply.

21. The device according to claim 16, further comprising an inductive coupling.

22. The device according to claim 16, wherein the memory is a non-volatile memory.

23. The device according to claim 16, wherein the at least one logic module is operable for executing programmable logic.

24. The device according to claim 16, wherein the indicator presents the first or the second state so as to be noticeable for a user and/or the first or the second state is readable by means of the indicator and/or the first or the second state can be transmitted by means of the indicator.

25. The device according to claim 16, further comprising at least sensor operable for measuring at least one ambient condition.

26. The device according to claim 16, wherein the device is configured to be attached to the medical products.

27. The device according to claim 16, further comprising an activator for activating the at least one activatable reset unit.

28. A system for disinfecting medical products, comprising:
a device comprising:
at least one sensor configured to register a UV-C light dose of a UV-C light treatment;
at least one activatable reset unit configured to perform a memory reset of a memory of the device;
at least one indicator; and
at least one logic module operatively coupled to each of the at least one sensor, the at least one activatable reset unit, the memory of the device, and the at least one indicator, wherein the at least one logic module is operable to determine whether medical products have been disinfected in a prescribed way on the basis of a UV-C dose registered by the at least one sensor and to determine whether the at least one activatable reset unit has been activated, wherein, by the at least one logic module, the memory is configured to be placed in a first state corresponding to a medical product disinfected in the prescribed way, and in a second state corresponding to the memory reset via the activated at least one activatable reset unit, wherein the state of the memory is externally communicated by the at least one indicator; and
an activator for activating the at least one activatable reset unit.

29. The system according to claim 28, wherein the activator is a transmitter.

30. The system according to claim 28, wherein the device is attachable to or configured to be incorporated into the medical product.

31. The system according to claim 28, wherein the at least one logic module is further configured to communicate with a disinfecting device during a disinfecting process performed by the disinfecting device.
